(19) United States
(12) Patent Application Publication
(10) Pub. No.: US 2007/0140909 A1
(43) Pub. Date: Jun. 21, 2007

(54) Biogas Analysis Tool (BAT)

(75) Inventor: Phillip Lewis Thompson, Seattle, WA (US)

Correspondence Address: Phillip Thompson, Seattle University, 900 Broadway - 524 ENGR, Seattle, WA, 98122, US

(73) Assignee: Phillip Lewis Thompson, Seattle, WA (US)

(21) Appl. No.: 11/306,282

(22) Filed: Dec. 21, 2005

(51) Int. Cl. G01N 33/00

(52) U.S. Cl. 422/83; 435/287.5

(57) ABSTRACT

The BAT can accurately and precisely measure the production of gas from microcosms containing bacteria. These microcosms are typically glass or plastic bottles (bioreactors) that contain bacteria and have an access portal in the form of a rubber septum. The needle of the BAT is inserted through the septum and the biogas travels through the BAT and displaces water in the burette. The change in the volume of water contained within the burette corresponds to the biogas production.

### DRAWING DESCRIPTION

FIG. 1 is a 2-dimensional drawing that illustrates the components of the Biogas Analysis Tool (BAT). It is comprised of a syringe-style needle that is connected to a luer-lock adapter. The adapter is connected to a gas-tight valve which is connected to a standard laboratory burette via laboratory-grade vacuum tubing and tubing connectors.

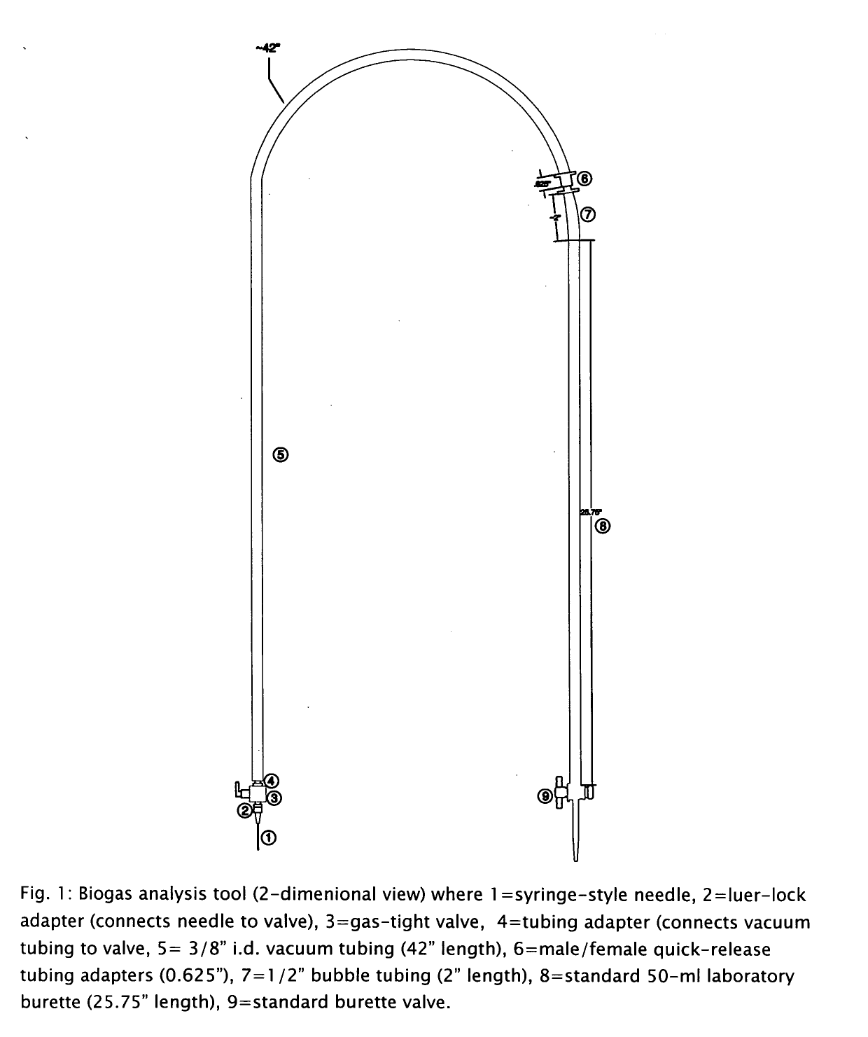

### INVENTION DESCRIPTION

The Biogas Analysis Tool (BAT) provides a means of measuring the production of small amounts of biogas via the displacement of water. The apparatus consists of a syringe-style needle which is attached with a luer-lock adapter to a gas-tight valve. The valve is connected to a standard burette with vacuum and bubble tubing.

What is claimed is:

1. The Biogas Analysis Tool is a unique device that can be used to measure small amounts (less than 50-ml) of gas production by aerobic, facultative or anaerobic bacteria. The BAT is particularly useful for assessing the stability of anaerobic digesters.
